(19) United States
(12) Patent Application Publication
(10) Pub. No.: US 2012/0193574 A1
(43) Pub. Date: Aug. 2, 2012

(54) Method Of Controlling Crystallization

(75) Inventors: Sharon Jane Cooper, Merryoaks (GB); Catherine Emma Nicholson, Peterlee (GB)

(73) Assignee: University of Durham, Durham (GB)

(21) Appl. No.: 13/393,523

(22) PCT Filed: Aug. 26, 2010
(86) PCT No.: PCT/GB10/01611

§ 371 Date: Apr. 19, 2012

(30) Foreign Application Priority Data
Sep. 1, 2009 (GB) 0915215.8
Jul. 5, 2010 (GB) 1011221.7

(51) Int. Cl. C07C 229/06; C09K 3/00; C07C 229/54

(52) U.S. Cl. 252/182.12; 562/553; 562/457

(57) ABSTRACT

The invention provides a method of crystallising a compound comprising either: (i) providing a first confined solution comprising the compound; and adding more of the compound to and/or increasing the degree of saturation of the first confined solution, whereby to provide a resultant second confined solution that comprises more compound and/or that has a greater degree of supersaturation relative to a confined supersaturated solution of the same compound stabilised solely by being confined; or (ii) providing a first confined melt comprising the compound; and cooling and/or increasing the pressure of the first confined melt, whereby to provide a resultant second confined melt that is cooler and/or is more pressurised relative to a confined super-cooled melt of the same compound stabilised solely by being confined, whereby to effect the crystallising under confinement and under thermodynamic control.

### FIELD

The present invention relates to a method for crystallisation, in particular, a method for crystallising in confined volumes under thermodynamic control. Through the method of the invention, thermodynamically favourable polymorphs may be provided, crystals obtained from systems that are difficult to crystallise and high quality nanocrystals provided, amongst other advantages.

### BACKGROUND

Control in crystallisation is hard to achieve because the crystallisation rate is typically governed by the energy barrier so that the kinetic product often crystallises initially. This is exemplified by Ostwald's 1897 rule of stages (W. Z. Ostwald, Phys. Chem., 1878, 22, 289), an empirical law in which it was noted that the least stable polymorph tends to crystallise first and then transform over time into more stable forms. Ostwald's rule is often, but not always, obeyed, and hence it is difficult to predict the polymorphic crystallisation outcome a priori.

For pharmaceutical companies, a practical effect of Ostwald's rule can be that it is not possible to determine whether the most stable polymorph of a (potential) drug has been found. This is a problem, because a more stable polymorph will have a lower solubility, and if the solubility of such a more stable polymorph is markedly reduced, this will significantly lower the rate at which the drug is absorbed resulting in a too-low dosage rate. The infamous case of the HIV-1 proteinase inhibitor Ritonavir (Norvir (Abbott), reported by S Datta and D J W Grant) Nature Reviews Drug Discovery, 3, 42-57 (January 2004)) exemplifies this. When it was first discovered in late 1992, ritonavir crystallised in a polymorphic form known as Form I. Other crystal forms of ritonavir were not discovered at that time. A New Drug Application was filed in 1995 and ritonavir was launched in 1996, formulated as soft gelatine capsules and as oral solutions. After failure of a dissolution test in early 1998, investigations revealed the existence of a less soluble, thermodynamically more favourable, polymorph. The lower solubility of this polymorph results in the precipitation of the drug and also a decrease in the dissolution rate of the marketed formulations. The adverse effect of the decreased dissolution rate on the bioavailability of ritonavir led to withdrawal of the remaining batches of ritonavir. A new formulation of ritonavir was developed, submitted to the FDA, approved and launched onto the market, but with considerable attendant expense.

Despite the potential disadvantages of incomplete analysis or understanding of polymorphic crystalline forms, the issue of predictability in relation to polymorph formation is often regarded as more of an art than a science. Indeed, an individual compound-by-compound experimental approach, such as iterative and/or high-throughput screening, is still arguably the most common methodology practised despite initiatives such as CPOSS (Control and Prediction of the Organic Solid State; www.cposs.org.uk), a basic technology programme with the aim of developing computational technology for the prediction of crystal structure(s) of organic molecules. Typically in the prior art, reports into control effected over polymorph formation tend to be limited to individual compounds, rather than being methodology of more general application. Thus, for example, U.S. Pat. No. 6,294,686 (Milhofer et al.) describes the preparation of new crystal forms of aspartame involving, in part, destabilisation of an aspartame-containing microemulsion. A. Kogan et al. (Langmuir, 2008, 24, 722-733) describe control over the crystallisation of the antiepileptic drug carbamazepine from various microemulsions, it being reported that the microstructure of the microemulsions influence the crystallisation process and so the polymorph obtained. This paper essentially describes how different polymorphs can be prepared from different microemulsions. This is achieved by interfacial crystallisation. J Yano, et al. (Langmuir, 2000, 16, 10,005-10,014) report on the crystallisation of glycine and I-phenylalanine from stabilised water-isooctane microemulsions with crystallisation induced by cooling to 5° C. after holding at 25 or 35° C. for one hour. K Allen et al. (Crystal Growth & Design, 2002, 2(6), 523-527) report on the crystallisation of glycine from aqueous solution within a variety of colloidal systems and describe the relationship between the polymorphic outcome and the nature of the extent of supersaturation.

The crystallisation of proteins is inherently difficult because of their varying molecular weights, shapes, aggregate states, and surface features that change with pH and temperature (for recent reviews see X. X. Li, et al. (Crystallography Reports, 2008, 53, 1261) and M. Caffrey (J. Struct. Biol. 2003, 142 108). Previous studies on precipitation of proteins from microemulsions have been conducted. However, the focus of these was on studies precipitating as much protein as possible in the shortest possible time (see D. G. Hayes and C. Marchio (Biotechnol. Bioeng. 1998, 59, 557); and J. Chen et al. (Colloid Surface B 2004, 33, 33)).

Common to much of the work hitherto reported (examples of which are referred to above), whilst aimed at achieving control over the polymorphic outcome of crystallisations, in particular from confined systems, efforts have not been made to achieve the most stable polymorph from the system but rather to simply achieve control per se. Thus investigations have tended to focus primarily on the nature of the systems in or from which crystallisation is achieved in order to control the polymorphic outcome, rather than by strictly controlling the crystallisation conditions.

More specifically, whilst crystallisation in confined volumes (such as microemulsions or emulsions) or restricted volumes (pores) has previously been shown to provide some selectivity over the crystallisation process so that either the most stable polymorph, or a metastable one, crystallises selectively, this selectivity has been attributed to specific interactions between the crystallising material and the confining material (i.e. the surfactant in the case of emulsions and microemulsions or the pore walls for crystallisation in mesoporous materials), or for microemulsion and emulsion crystallisation, the ability of the crystallising material to act as a co-surfactant, so that interfacial crystallisation occurs. However, no generic method for ensuring polymorphic selectivity in confined or restricted volumes has emerged, limiting the predictive capability of such techniques and their ability to be used more widely.

In view of the advantageousness of inter alia identifying the most stable polymorph of a given substance, for example to ensure reproducible bioavailability, or to achieve the most stable polymorphic outcome from any given system, it would be of general benefit to the art if any given crystallisation procedure was independent of the substance to be crystallised and/or knowledge as to any polymorphic variability.

### SUMMARY

We have surprisingly found that generic and predictive control may be achieved, if achievable, over crystallisations effected within confined volumes by providing either a supersaturated solution or a supercooled melt that is stabilised solely due to confinement and then relative to such a stabilised supersaturated solution or supercooled melt, adding more crystallisable material, or increasing the degree of supersaturation, or cooling or increasing the pressure of the supercooled melt whereby to obtain crystallisation under thermodynamic control. Thermodynamic control is achieved because of the scarcity of the crystallising material, as described in greater detail herein. The combination of the provision of a supersaturated solution or a supercooled melt, within confined volumes such as in a micro- or nanoemulsion, with effecting increase in the degree of supersaturation (or decreasing the temperature/increasing the pressure) of the confined regions within such systems, or adding more crystallisable material to such systems, permits predictable control over the polymorphic outcome of a crystallisation such that crystallisation from the system may be effected under thermodynamic control, contrary to Ostwald's rule of stages. If a given compound does not exhibit polymorphism, the present invention is none the less useful since the thermodynamic control under which crystallisation is effected is capable of delivering high quality crystals, e.g. high quality nanocrystals.

Viewed from a first aspect, the invention provides a method of crystallising a compound comprising either:
(i) providing a first confined solution comprising the compound; and adding more of the compound to and/or increasing the degree of saturation of the first confined solution, whereby to provide a resultant second confined solution that comprises more compound and/or has a greater degree of supersaturation relative to a confined supersaturated solution of the same compound stabilised solely by being confined; or
(ii) providing a first confined melt comprising the compound; and cooling and/or increasing the pressure of the first confined melt, whereby to provide a resultant second confined melt that is cooler and/or is more pressurised relative to a confined supercooled melt of the same compound stabilised solely by being confined,
   whereby to effect the crystallising under confinement and under thermodynamic control.

Viewed from a second aspect, the invention provides the use of a solution or melt comprising a compound in a method of crystallising the compound, comprising a method according to the first aspect of the invention.

### BRIEF DESCRIPTION OF THE FIGURES

FIG. 1 shows a graph of Helmholtz free energy of formation ΔF of a nucleus of radius, r. The ΔF*, r* and r₀ values are indicated.

FIG. 2 shows a graph of Helmholtz free of energy of formation ΔF of polymorphic crystalline forms a and b from a melt, in which Oswald's rule of stages is obeyed. Polymorph b is less stable than polymorph a.

FIG. 3 shows a graph of Helmholtz free of energy of formation ΔF of polymorphic crystalline forms a and b from a solution in a nanoconfined volume, in which Oswald's rule of stages is obeyed. Polymorph b is less stable than polymorph a.

FIG. 4 depicts seven representative ATR (attenuated total reflection) FTIR spectra from Example 1 showing the excellent reproducibility in obtaining the stable γ-polymorph of glycine with virtually no α-polymorph, with the relative proportions of each polymorph indicated by the relative heights of the asterisked peaks at 928 cm⁻¹ (γ) and 910 cm⁻¹ (α).

FIG. 5 depicts a representative selected area electron diffraction pattern from Example 2 below showing the hexagonal γ-polymorph of glycine obtained from nanocrystal aggregates.

FIG. 6 depicts a representative ATR (attenuated total reflection) FTIR spectrum from Example 3 below showing predominantly the γ-polymorph of glycine with some α-polymorph, with the relative proportions of each indicated by the relative heights of the asterisked peaks at 928 cm⁻¹ (γ) and 910 cm⁻¹ (α).

FIG. 7 depicts a representative ATR FTIR spectrum from Example 5 below showing the characteristic 2231 cm⁻¹ peak for the yellow prism polymorph of ROY, and absence of any other characteristic peaks for the other ROY polymorphs in this region.

FIG. 8 depicts a representative spectrum obtained from Raman spectroscopic analysis of mefenamic acid crystals produced in accordance with Example 4. This shows the characteristic Form I peaks at 624 and 703 cm⁻¹ and the absence of peaks at 631 and 694 cm⁻¹ that are characteristic of Form II.

### DETAILED DESCRIPTION

The present invention provides inter alia the ability to prepare the most thermodynamically stable polymorph or polymorphs of a compound where the possibility of different polymorphs exists, i.e. the compound exhibits polymorphism. As is known, polymorphs are crystalline substances that have different internal crystal structures but which have the same chemical composition.

According to certain embodiments of the invention, there is provided a method of crystallising a compound comprising providing a supersaturated solution or supercooled melt comprising the compound, the supersaturated solution or supercooled melt being stabilised solely by being confined and then adding more of the material (i.e. the compound) and/or increasing the degree of supersaturation of the supersaturated solution, or cooling or increasing the pressure of the supercooled melt, whereby to effect the crystallising under confinement and under thermodynamic control. There is also provided the use of a supersaturated solution or supercooled melt comprising a compound, the supersaturated solution or supercooled melt being stabilised solely by being confined, in a method of crystallising the material (i.e. the compound) comprising adding more of the compound and/or increasing the degree of supersaturation of the supersaturated solution, or cooling or increasing the pressure of the supercooled melt, whereby to effect the crystallising under confinement and under thermodynamic control.

The first step in the practice of these embodiments of the present invention is to provide a supersaturated solution or supercooled melt that is stabilised solely due to confinement.

As is known in the art, a supersaturated solution is a solution comprising more solute than that which could be introduced into the solvent of the solution under the same conditions. Likewise, a supercooled melt refers to molten material that could not be prepared by exposure of a solid material to the same conditions in which a supercooled melt exists.

Supersaturated solutions can, in general terms, be provided by one of the following:
effecting temperature change (typically cooling) to a saturated (or undersaturated) solution;
adding antisolvent (i.e., defining antisolvent generally, a liquid miscible with the solvent in a solution but which is not, itself, a good solvent for the solute or one of the solutes, i.e., typically the solubility of a or the solute (e.g. the compound according to the first aspect of this invention) is lower in the antisolvent than in the solvent of the first confined solution of the first aspect of this invention) to a saturated (or undersaturated) solution;
evaporation of solvent from a saturated (or undersaturated) solution; or
effecting a chemical reaction in which the products are less soluble than the reactants.

Typically, supersaturation may be achieved by effecting temperature change or antisolvent addition to a saturated (or undersaturated) solution.

Most, but not all, solutes have decreased solubility on cooling. For this reason, if a saturated or undersaturated solution comprising a typical solute is cooled to below the temperature at which a solution of that concentration is saturated, then a supersaturated solution may be generated. Conversely, if a solute has a solubility that increases on cooling, a supersaturated solution may be provided by increasing the temperature of a saturated or undersaturated solution. Analogously, when supersaturation is achieved by antisolvent addition, an initial saturated (or undersaturated) solution may be provided into which antisolvent may be added.

The preparation of supercooled melts may be achieved by subjecting an existing melt either to temperature and/or pressure change (typically, but not necessarily, cooling and/or an increase in pressure). Typically, the preparation of supercooled melts is achieved by cooling molten material beneath its freezing point rather than through the (optionally additional) use of pressure.

In some of the discussion that follows hereinafter, reference is made to embodiments of the invention that involve use of systems comprising supersaturated solutions as opposed to supercooled melts. However, the invention is not to be construed as being so limited.

Those of skill in the art are aware of the concept of crystallisation within confined spaces. This concept is, for example, described in a number of the documents identified in the Background section above, and references cited therein. Examples of systems within which confined supersaturated solutions or supercooled melts may be prepared, and in which a supersaturated solution or supercooled melt is stabilised solely due to confinement, include microemulsions, nanoemulsions and liposomes. For the avoidance of doubt, whilst crystallisation in confined spaces is a concept well-understood to those of skill in the art and microemulsions, nanoemulsion and liposomes are likewise well-understood terms of the art, the following definitions, including of particular systems within which certain embodiments of the invention may be practised, and a related term (emulsion), are set forth to assist with understanding the present invention as typical features of representative examples of confined systems within which supersaturated solutions may be prepared:

A stable state refers to a system which is in equilibrium (i.e. not changing with time) and in its lowest energy state, so the system is at a global energy minimum. The system will persist in this state indefinitely, unless the system is changed. A metastable state refers to a system which is in equilibrium but which is susceptible to fall into a lower energy state with only a slight perturbation, i.e. the system is in a local energy minimum but not the global one.

Crystallisation can conveniently be considered to occur in two stages, nucleation and then crystal growth. For crystallisation to occur, a solution must be supersaturated, or the melt supercooled, so that there is a thermodynamic driving force for crystallisation to occur. In addition, an energy barrier must then be overcome, owing to the interfacial area that has to be created in forming the new phase. The process of surmounting this energy barrier is known as nucleation, and the nucleus of the new phase corresponding to the energy maximum is known as the critical nucleus, having radius r*. After nucleation, crystal growth occurs as additional crystallisable molecules attach to the nucleus, until the supersaturation/supercooling of the system is relieved.

Emulsions: these are thermodynamically metastable, cloudy mixtures of immiscible liquids, for example oil-in-water or water-in-oil emulsions in which the droplet size of the discontinuous phase (also referred to as the dispersed phase) may be from about 500 nm to several pm in one dimension (typically diameter). Droplets may be stabilised due to the presence of surfactants, optionally, in combination with co-surfactants. Dissolved solutes may be present within the continuous and/or discontinuous phases.

Microemulsions: these are thermodynamically stable, transparent, mixtures of immiscible liquids, for example oil droplets in water (an oil-in-water microemulsion) or water droplets in oil (a water-in-oil microemulsion). The droplet size is typically less than 50 nm in diameter, for example of approximately 1 or 2 nanometres to 50 nanometres in at least one dimension (typically diameter). Droplet sizes are typically about 2 to 10 nm. Typically, there is a relatively narrow polydispersivity of σ_{R}/Rₘₐₓ, where σ_{R} is the standard deviation and Rₘₐₓ is the modal droplet radius (J. C. Eriksson and S. Ljunggren, Langmuir, 11, 1145-1153 (1996)). The droplets are stabilised on account of the presence of surfactants, frequently in combination with a co-surfactant, which reside at the droplet interface. When the volume fraction of the dispersed phase becomes so low that its properties differ measurably from its usual bulk properties, the terms “swollen micelles”, “swollen micellar solutions”, “solubilised micellar solutions” or even simply “micellar solutions” can be used instead of microemulsions for oil-in-water systems, whilst for water-in-oil systems, the same terms but with “inverse” or “reverse” inserted before “micelle” or “micellar” may be used. However, because there is, in general, no sharp transition from a microemulsion containing an isotropic core of dispersed phase and a micelle progressively swollen with the dispersed phase, many researchers use the term “microemulsion” to include swollen micelles (or swollen inverse micelles) but not micelles containing no dispersed phase. This is the context in which the term “microemulsion” is used here. In the microemulsions, dissolved solutes (to give rise to a desired supersaturated solution) may be present within the discontinuous (dispersed) phase (i.e. the oil droplets in oil-in-water microemulsions) giving rise to the supersaturated solutions stabilised solely due to confinement of utility in the present invention. Alternatively or additionally, solute may be present in the continuous phase. The dispersed phase in a microemulsion may constitute a supercooled melt stabilised solely by being confined of utility in the present invention.

Nanoemulsions (sometimes known as miniemulsions): these are thermodynamically metastable, blueish transparent or blueish white translucent mixtures of immiscible liquids, for example oil-in-water or water-in-oil emulsions in which the droplet size of the discontinuous phase may be from about 50 to 500 nm, typically about 50 to 100 nm in one dimension (typically diameter). As with microemulsions, the droplets are stabilised due to the presence of surfactants, optionally, in combination with co-surfactants. Dissolved solutes may be present within the discontinuous phase giving rise to the supersaturated solutions stabilised solely due to confinement of utility in the present invention. Alternatively or additionally, solute may be present in the continuous phase. The dispersed phase in a nanoemulsion may constitute a supercooled melt stabilised solely by being confined of utility in the present invention.

A co-surfactant is a surfactant (i.e. a compound that adsorbs at surface or interfaces) that is used in combination with, and serves to enhance the effectiveness of, another surfactant. Co-surfactants are often used when preparing emulsions, such as micro- or nanoemulsions. Common co-surfactants are alcohols comprising about 3 to 8 carbon atoms.

Homogeneous nucleation refers to the initial stage of crystallisation (i.e. the nucleation) occurring in the interior of the sample and not upon a surface. The homogeneous nucleation temperature is the temperature at which the crystallisation is typically observed to occur on cooling a sample following homogeneous nucleation. To ensure that the nucleation does not occur upon a surface, experiments to determine homogeneous nucleation temperatures can be conducted in emulsions of μm size, from about 500 nm to several pm, for example, since these are sufficiently small to exclude foreign particles such as dust, provided the surfactant or co-surfactants used do not aid nucleation.

Heterogeneous nucleation is when the nucleation occurs upon a foreign surface. In the present invention, heterogeneous nucleation can occur if the crystallisable molecules adsorb sufficiently onto the surfactant or co-surfactants, to cause nucleation at this boundary.

It is well known that some surfactants or co-surfactants can assist with, or induce, crystallisation by aiding the nucleation. For example addition of heptacosanol to a water-in-oil microemulsion (in which the water droplets may be regarded as regions of melt) can induce ice crystallisation, on account of the similarity of the crystal structure of long-chain alcohols to ice. It is straightforward to verify whether a surfactant or co-surfactant aids nucleation of a particular polymorph by, for instance, determining whether preferential crystallisation of a particular polymorph occurs at the planar air-water or oil-water interface in the presence of these same surfactants or co-surfactants.

Liposomes: as are also well understood, liposomes are typically spherical particles within an aqueous medium constituted by a lipid bilayer enclosing an aqueous compartment. Liposomes are typically of the order of tens of nanometres in diameter (for example about 10 to about 100 nanometres in diameter).

Other confined systems within which supersaturated solutions or supercooled melts may be prepared are known to those of skill in the art. Examples include distributed confined solutions within polymeric matrixes of polymeric gels, or within mesophases such as lyotropic liquid crystal phases. Typically, however, because of the ease of providing useful homogeneity of distribution of the combined supersaturated solutions or supercooled melts; and/or homogeneity of the encapsulated voids within which the supersaturated solutions or supercooled melts are confined and/or the occurrence of 3D nano-confinement, microemulsions and nanoemulsions, and in particular microemulsions, are described in particular detail herein in connection with particular embodiments of the invention. The invention is, however, not to be understood to be so limited.

Microemulsions in particular are convenient vehicles to study crystallisation in confinement because they are thermodynamically stable, have a relatively monodisperse droplet size that can be easily varied by altering the composition, the width of premelting/unfreezable layers can be known reliably, and the supersaturation is little affected by the confinement owing to the negligible LaPlace pressure difference across the interface (A. Sanfeld et al., Adv. Coll. Interface Sci. 2000, 86, 153). In addition, confinement can be assumed when the droplets are non-percolating, i.e. the droplets tend not to aggregate (verifiable by e.g. conductivity and dynamic light scattering measurements (see e.g. P. Tartaglia et al., Phys. Rev. A 1992, 45, 7257)).

A particular feature of certain aspects of the present invention is that the supersaturated solutions or supercooled melts, where provided for use in the present invention, in particular confined supersaturated solutions or supercooled melts, are present in a state that can be shown to be stabilised solely due to confinement, a phrase used interchangeably herein with stabilised solely by being confined. By this is meant that they may remain in this state without crystallising for some time, for example at least a week, more typically at least a month. The stabilities of the confined supersaturated solutions or supercooled melts provided for use in the present invention are always much longer than the corresponding unconfined systems, which would crystallize quickly, for example within hours or minutes. By confinement (or confined) is meant herein that the supersaturated solution or supercooled melt (that is confined) is wholly surrounded and so encapsulated by a (typically non-gaseous) material, other than the supersaturated solution or supercooled melt. It will be immediately understood how this is so with systems such as micro- and nanoemulsions and with liposomes in which droplets of supersaturated solution or supercooled melt may be formed as either regions of discontinuous phase within such micro- or nanoemulsions; or within lipids in liposomes. Because of the dimensions of the volumes of confinement in these systems, discussed infra, the term nanoconfinement is used herein to indicate confinement within the confined regions of systems of such dimensions (i.e. of about 1 nm to about 500 nm in at least one dimension), for example confinement within the dispersed phase in microemulsions.

Crystallisation of polymorphic (or potentially polymorphic) material within confined volumes is under thermodynamic control according to this invention. This is because of the limited amount of material within the confined volume.

Those of skill in the art will be aware that crystals grown within droplets of micro- or nanoemulsions, for example, can grow, with time, to sizes much larger than the initial droplet sizes, showing that the confinement is not permanent. This arises because the microemulsion droplets are in constant motion (Brownian motion) and undergo collisions with one another. The most energetic collisions between two droplets can lead to a transient dimer forming, during which short time material within the interior of the droplets can be exchanged. If one of the colliding droplets contains a crystal nucleus, its surrounding solution/melt has a lower concentration of crystallisable material than a droplet without a nucleus in it, and so crystallisable molecules will tend to flow out of the more concentrated region into the less concentrated region and then attach onto the crystal nucleus, thereby causing it to grow. This process can be repeated until eventually the crystal becomes larger than the droplet which originally confined it. Additionally, if a transient dimer forms between two droplets that each contain nuclei, the two nuclei can combine by oriented attachment to form a single larger nucleus. Provided the energy barrier to nucleation is surmountable, the relative probability that a droplet contains a nucleus depends upon the difference in free energy, ΔF, of the nucleus compared to the mother solution, or melt, and is given by the Boltzmann factor exp(−ΔF/kT) where k is the Boltzmann constant and T is the temperature. Thus any nuclei that can form with ΔF≦0 (i.e. stable nuclei) will be prevalent, whereas those with ΔF≧0 will be far fewer in number and short-lived.

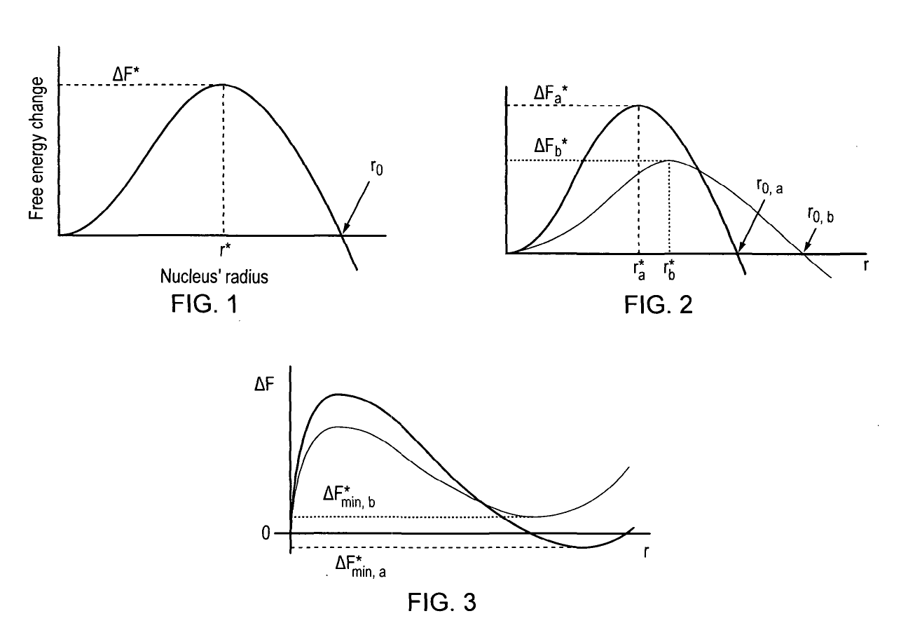

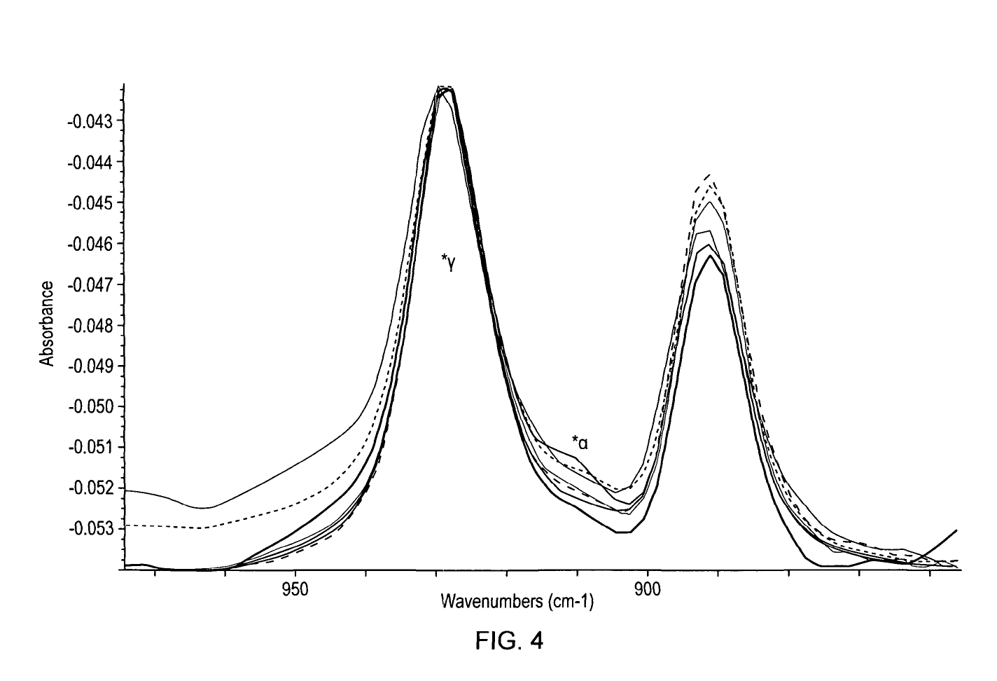

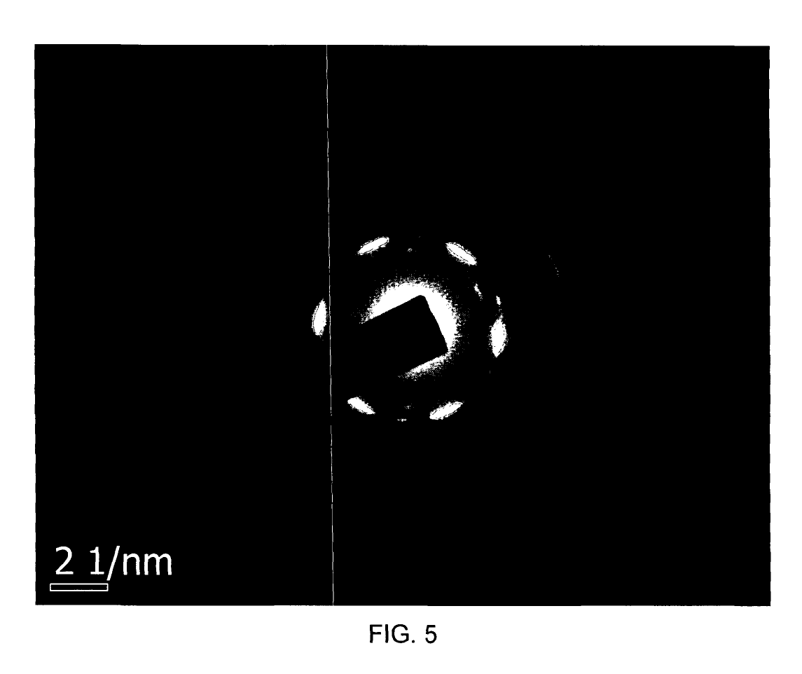

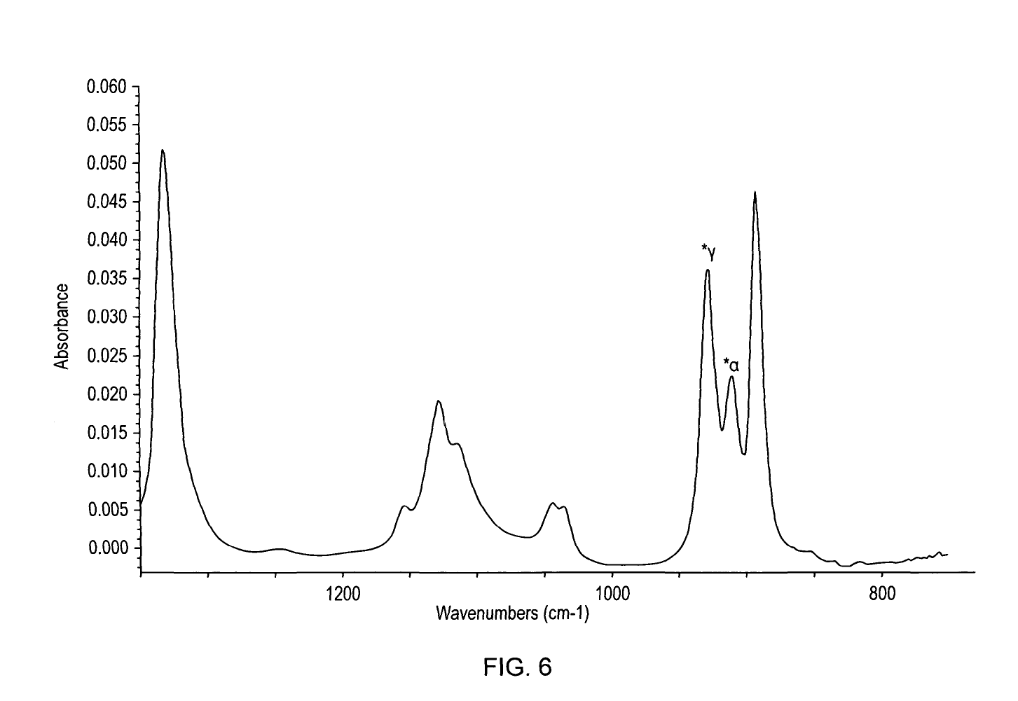

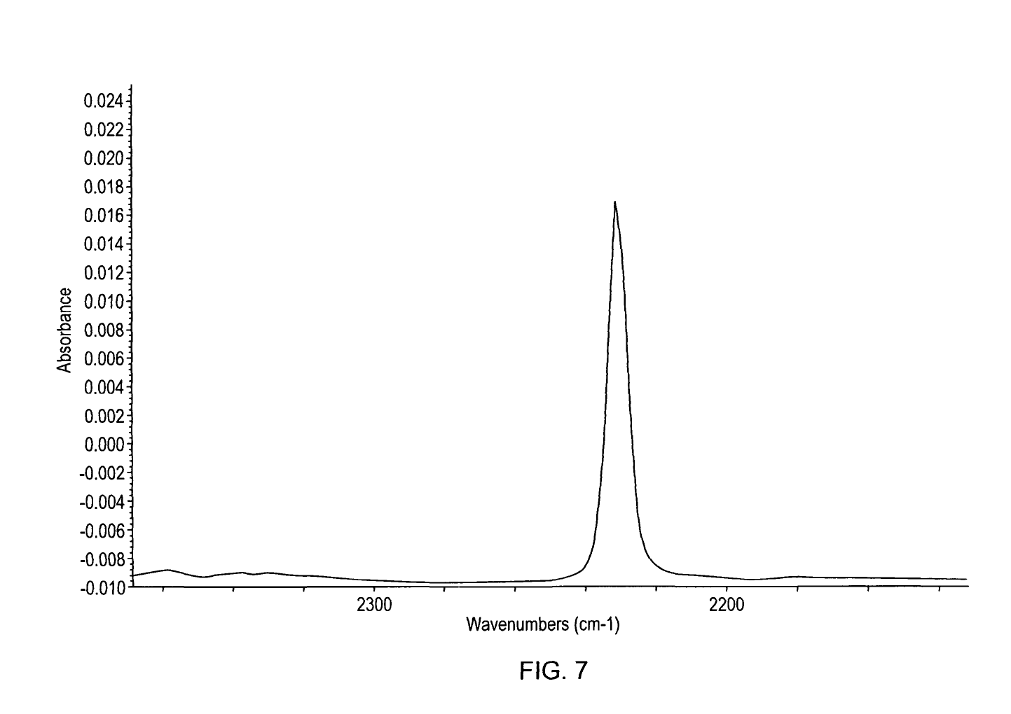

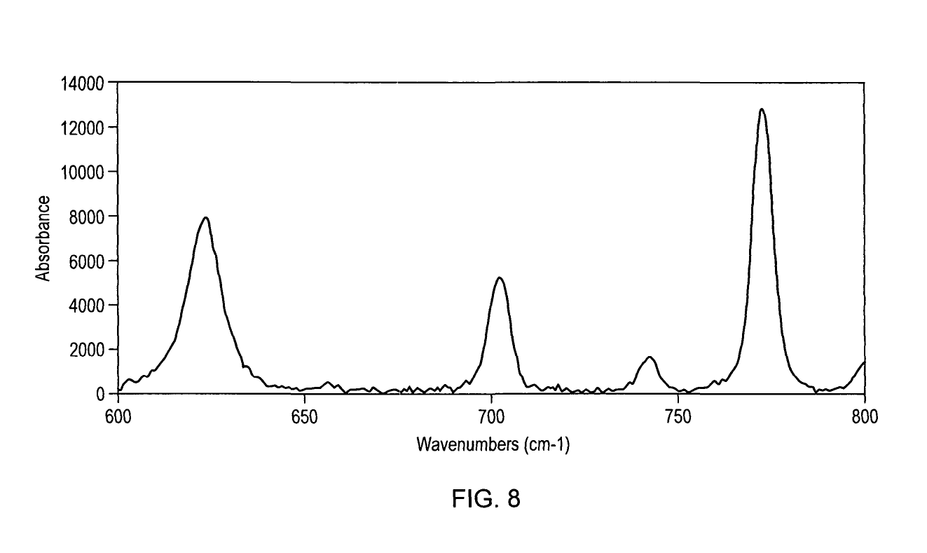

In accordance with embodiments of the present invention, supersaturated solutions or supercooled melts are prepared that are stabilised solely due to confinement. This stability arises, in part, because, whilst the material within the system may have enough energy to obtain the critical nucleus size r*, there is insufficient material in order to grow to the minimum stable nucleus size r₀. This is depicted schematically in FIG. 1, which is an exemplary graph of the Helmholtz free energy of formation ΔF of a nucleus of radius r. The maximum free energy ΔF*, r*, and r₀ values are indicated. A supersaturated system stabilised solely due to confinement would be one where nuclei of size r could form, where r*<r<r₀ but not r≧r₀.

FIG. 1 shows the Helmholtz free energy of formation of nuclei of radius r in the absence of (at least indicated) polymorphism, for crystallisation from a melt. Where the free energy barrier ΔF* is surmountable, and there is just sufficient material to crystallise a rₒ crystallite, the phase transition from melt to crystalline form is thermodynamically feasible and crystallisation will occur. In other words, in this specific case, the phase transition temperature will be controlled by the requirement that ΔF=0 on complete crystallisation of the confined system

FIG. 2 shows another graph of Helmholtz free energy of formation of nuclei of radius r from the melt, but this time reflects the existence of polymorphic forms a and b. The graph indicates that polymorph a is more thermodynamically stable and polymorphic form b is less stable (i.e. less thermodynamically stable) at least in respect of the case of crystallisation from the melt reflected in the graph. In this system, polymorph b will typically crystallise first (in accordance with Ostwald's rule) and the existence of the polymorphic form a may never be known.

A crystallising system obeying Ostwald's rule, such as that depicted in FIG. 2, has a higher energy barrier to nucleation for its stable polymorph than its metastable one. However, if the crystallisation is conducted not in an unconfined volume, where the formation of nuclei of radius r_{0,a} or r_{0,b} are possible, but instead in a confined volume engineered such that there is only sufficient material to form a nucleus of radius r_{0,a}, then because there is insufficient material to form polymorph b having r_{0, b}, only polymorph a will form, because only polymorph a can form a new stable phase. In other words, crystallising in a confined volume such that only nuclei of size r can form where r_{0,a}≦r<r_{0,b} is sufficient to ensure that only the stable polymorph a nucleates, and thermodynamic control of crystallisation is thereby achieved.

In this way, with reference to the discussion above, stability solely due to confinement will correspond to the scenario depicted in FIG. 2, where it is possible for nuclei of critical size r*ₐ and r*_{b} to form but, because of the scarcity of material, nuclei cannot grow to a size r_{0,a} or more.

By operating under conditions whereby the nucleation barrier is surmountable, but the amount of material within each confined region is limited, then thermodynamic control can be achieved when only a single stable nucleus can form within each region of confinement, with this stable nucleus necessarily comprising the lowest possible energy form(s). The resulting crystalline product will then reflect the relative population of the lowest energy post-critical nuclei that can form within the confined droplets. This differs markedly from crystallisation under kinetic control, whereby the crystalline product first formed reflects the relative population of the highest energy nuclei (i.e. the critical nuclei) of each polymorph. Hence, with reference to FIG. 2, if there is insufficient material to form an r_{0,b} nucleus, but an r_{0,a} nucleus can form, thermodynamic control is achieved and polymorph a will crystallise. In contrast, if crystallisation is under kinetic control, e.g. in an unconfined system, polymorph b will crystallise, as r*_{b} is lower in free energy than r*ₐ.

Thermodynamic control is also possible according to many embodiments of this invention in allowing crystallisations to be achieved from supersaturated solutions contained within appropriately confined volumes. In these embodiments, a minimum in the free energy arises owing to the decrease in solute concentration, and thus supersaturation, as the new crystal phase grows. Thermodynamic control is then achieved by selection of an appropriately confined volume size such that the free energy minimum value, ΔF*ₘᵢₙ<0 for polymorph a, but not b. This is shown schematically in FIG. 3.

In certain embodiments of the invention, therefore, micro- or nanoemulsions comprising dispersed regions of supersaturated solution or supercooled melt may be prepared in a convenient manner by effecting temperature change or by addition of antisolvent as hereinbefore described. Where a supersaturated solution is achieved for example by way of addition of antisolvent, the micro- or nanoemulsion is typically prepared with the (more) effective solvent for a solute, with supersaturation achieved through vapour diffusion of the antisolvent into the micro- or nanoemulsion by placing the micro- or nanoemulsion in a container into which the antisolvent evaporates. Alternatively, addition may be of a separate microemulsion or nanoemulsion or emulsion containing the antisolvent in its confined phase. As a further alternative, the antisolvent can be added dropwise to the micro- or nanoemulsion, although care must be taken in this latter embodiment, for example by limiting the size of the drops, to avoid too high local concentrations of antisolvent, since these can potentially lead to destabilisation of micro- or nanoemulsion droplets and consequential coalescence before and/or during crystallisation, leading to loss of ability to effect the desired thermodynamic control under which crystallisation is effected in accordance with the present invention. Dropwise addition of antisolvent may also cause the supersaturation of the system to increase too much, so that stable nuclei of many different forms can form in each droplet in which case thermodynamic control may again be lost, if the crystallisation is governed by the rate at which these stable forms can form, and not their inherent stability.

It is important to note that the solubility of a solute in a micro- or nanoemulsion can, as the skilled person is aware, differ significantly from the corresponding solubility in bulk solution. For example, the solubility can decrease substantially in confined solutions if some of the solvent is bound to the surfactant, resulting in it being unavailable to assist in the dissolution of the solute. Alternatively, a solute's solubility, and thus its concentration within the dispersed droplets in a micro- or nanoemulsion may increase substantially if the solute itself is surface-active (for example because it has both hydrophilic and hydrophobic regions) so that it adsorbs significantly (in the context of a confined dispersed phase having disproportionately high surface area vis-à-vis a bulk solution) at the oil/water interface. For instance, substantial uptake of water into the surfactant layer occurs for nonionic surfactants with multiple ethylene oxide groups, with ˜1-3 water molecules per ethylene oxide group being used to hydrate the surfactant (Y. Feldman et al., Colloids and Surfaces A: Physicochemical and Engineering Aspects, 128 1997 47; S. Ezrahi et al., J. Dispersion Sci. Technol. 2002, 23, 351), whilst if the solute has co-surfactant properties it may absorb in this interfacial region to a significant extent, as found for the antiepileptic drug, carbamazepine (A. Kogan, A. Aserin, N. Garti J. Coll. Inter. Sci, 2007, 315, 637), and the artificial sweetener, aspartame (H. Furedi-Milhofer, N. Garti, A. Kamyshny, J. Cryst. Growth, 1999, 198, 1365).

For this reason, it is important to ascertain initially the solubility of a solute in a confined solution, such as a micro- or nanoemulsion. This is easily achievable by those skilled in the art by simply adding a very small quantity (approximately mg quantities) of the solute to a micro- or nanoemulsion at the temperature of interest and leaving it to dissolve over a few days or weeks until no more solute can be dissolved within the system.

It is also important to note that if the solubility of the solute is significantly reduced in a micro- or nanoemulsion, or other confined system, as compared with the corresponding bulk solution, it is possible that when the confined system is prepared the solute can be present in supersaturated dispersed regions. This may of course be tolerable as long as the degree of supersaturation is not such a degree that immediate and uncontrolled crystallisation occurs: if this is the case, then crystallisation under thermodynamic control is unlikely to occur, not least since such a supersaturated system is clearly unstable, let alone stabilised solely by being confined. The generation of unstable micro- or nanoemulsions is easily avoidable and/or may be determined by the skilled person. To assist in the provision of appropriate supersaturated solutions or supercooled melts that have utility in the present invention, the skilled person is aware of the following formulae:
supersaturation is often quantified by the supersaturation ratio=c/cₛₐₜ wherein c is the concentration of the compound in the microemulsion and cₛₐₜ is the saturation concentration in the microemulsion; and for supercooled melt: supersaturation is often quantified by the undercooling=(Tₘ−T), wherein Tₘ is the melting temperature.

The preparation of confined systems having suitable size may be readily accomplished by those of skill in the art. It may be convenient to measure the confinement size (e.g. droplet size of the microemulsions). This may be achieved, for example, by small angle X-ray scattering, small angle neutron scattering, or dynamic light scattering. However a rough estimate of droplet size, assuming the droplets are spherical and the vast majority of the surfactant resides at the droplet interface is given by the equation:

droplet radius R=3*volume fraction of internal phase/total interfacial area per unit volume.

The volume fraction of the internal phase includes the polar phase (e.g. water and everything dissolved within it) and the polar/ionic headgroups of the surfactant for a water-in-oil microemulsion. For an oil-in-water microemulsion, the internal phase comprises the non-polar phase (i.e. oil and everything dissolved within it) and the non-polar tailgroups of the surfactant. The total interfacial area per unit volume is the interfacial area of the surfactant molecule (typically estimated from experiments using the surfactant at the planar air-water or oil-water interface) multiplied by the number of surfactant molecules per unit volume. It may therefore be appreciated that the more internal phase added relative to the surfactant, the larger the droplet size is likely to be, although if too much internal phase is added a microemulsion may not form. This is because, as is known, surfactants enable microemulsions as a class to form by having an ultra-low interfacial tension between the water and oil phases, which can only be maintained if there are sufficient surfactant/co-surfactant molecules present to cover the whole of the oil-water interface. The maximum achievable droplet size for a microemulsion depends upon the surfactants/co-surfactants used.

It should be noted that the preparation of a supersaturated solution in a microemulsion is not a sufficient condition to achieve thermodynamic control of crystallisation because the supersaturation may not be high enough for the crystallisation to occur (i.e. the energy barrier to crystallisation is not surmountable) or the droplets may already contain enough material to form stable nuclei of more than one polymorph. Nor is it possible to generally start with extremely small droplets and increase the supersaturation/supercooling until crystallisation occurs, as the microemulsion may become unstable and hence the confinement lost prior to the crystallisation commencing, or else the supersaturation at which only one stable polymorph can form within the droplet may be passed through too quickly to allow crystallisation of just this polymorph. Consequently it is advantageous to establish that a state will have been achieved, prior to the crystallisation, where the supersaturated solution/supercooled melt is stabilised solely due to confinement. Alternatively, once a protocol is established that provides such a state, a state of identical composition may be prepared on which crystallising under confinement and under thermodynamic control may then be effected.

A preferred embodiment for achieving thermodynamic control of crystallisation in accordance with the present invention is therefore illustrated by the following, non-limiting steps (described with reference to those embodiments of the invention making use of stabilised, confined supersaturated solutions):
(1) Create an undersaturated solution. This ensures that no nuclei are present;
(2) Create a STABILISED, supersaturated solution within a confined volume. Such a stabilised supersaturated solution will typically not crystallise in less than a week, and typically less than a month, since there is insufficient material to allow crystallisation;
(3) Add more material, typically incrementally, to the confined volume and/or increase the supersaturation to induce crystallisation in the confined volume. Induction of crystallisation may either be effected to a system shown to be stabilised solely due to confinement, or, for example, by making a fresh microemulsion sample, with incrementally more material or supersaturation compared to the system stabilised solely due to confinement;
(4) ALWAYS achieve thermodynamically most stable polymorp crystals with improved quality/stability.

It is possible to demonstrate the provision or existence of a supersaturated solution that is stabilised solely due to confinement in practice by demonstrating that such a system does not crystallise whereas, counterintuitively, a corresponding system containing the same constituents but having larger volume of confinement (e.g. droplet size within the nanospaces of a micro- or nanoemulsion containing a crystallising material (e.g. solute within the dispersed/discontinuous phase within the micro- or nanoemulsion)), at a lower concentration level, does. Demonstration of this provides the necessary and sufficient evidence to establish that a system suspected of being at, or comprising regions of, supersaturated solution stabilised solely to confinement is indeed such a system. This is because such a test demonstrates that the crystallisation has not been limited by the energy barrier to the process, since this has been increased for the system which did crystallise (since the solute concentration and so extent of supersaturation was lower), but instead by the scarcity of the crystallising material. In a supersaturated system that may, if convenient, be thereby demonstrated to be stabilised solely due to confinement, crystallisation can then be effected under thermodynamic control, typically incrementally, either as a consequence of the addition of more crystallisable material, or by way of an alternative method of increasing the extent of supersaturation of the system (e.g. by addition of antisolvent), again typically incrementally. Crystallisation may be effected in this way either on a system demonstrated to be stabilised solely due to confinement, or on a fresh system of identical composition.

Analogously, a supercooled melt that is stabilised solely due to confinement may be demonstrated by showing that a corresponding system with the same constituents, only, for example, with larger sized droplets within a micro- or nanoemulsion crystallises at or above the same temperature as that at which the test micro- or nanoemulsion thereby demonstrated to be stabilised solely due to confinement does not.

In practice, a supersaturated solution or supercooled melt may be demonstrated to be stabilised solely due to confinement in a number of ways. Importantly, however, such systems must be free of any prior crystallised species when they are prepared, i.e. actually be supersaturated solutions or supersaturated melts. For this reason it is advantageous to prepare the microemulsion or nanoemulsion using undersaturated solutions or stable melts, and then to induce the supersaturation afterwards by cooling/antisolvent addition.

If supersaturated solutions or supercooled melts are used to prepare the microemulsion or other confined systems, the skilled person will be aware that transparency per se of micro- or nanoemulsions or other systems capable of providing approximately confined cavities is not sufficient to verify the absence of crystallisation. This is because any crystalline particles could be too small to scatter light. For this reason, an additional test may be appropriate in embodiments in which a microemulsion or nanoemulsion has been prepared from supercooled melts or supersaturated solutions in order to verify the absence of any previous crystallisation, and so demonstrate a putative supersaturated solution or supercooled melt to actually be such. A simple example of such a test would be to demonstrate that any supersaturated solution or supercooled melt left over after the microemulsion or nanoemulsion has been prepared remains in this metastable state over one day.

Other tests for the pre-existence of crystallise material include transmission electron microscopy, X-ray diffraction and nuclear magnetic resonance spectroscopy adapted to detect crystallite sizes of 2 nm and above. Such methods are known in the art.

Thus, it may, in certain embodiments of the invention, be appropriate to prepare two microemulsions (or nanoemulsions or liposomes) with supersaturated solutions, or two microemulsions (or nanoemulsions or liposomes) with supercooled melts. Typical methods of forming these microemulsions with supersaturated solutions or supercooled melts would be by using undersaturated solutions or melts above their melting temperature, with the supersaturation or supercooling then achieved after the microemulsions are formed, since this ensures that no pre-existing nuclei are present within the microemulsions. The supersaturation or supercooling can readily be achieved after the microemulsions are formed by temperature change or addition of antisolvent, or may occur at the instance of microemulsion formation if solvent is taken up by the surfactant/co-surfactant used to prepare the microemulsion. Alternatively if supersaturated solutions or supercooled melts are used to form the microemulsions initially, then tests for the absence of pre-existing nuclei in the supersaturated solutions/supercooled melts may be undertaken as described above. Where microemulsions containing supersaturated solutions are used, one of the systems will contain larger droplets but have a lower concentration of crystallisable material within the droplets. Where microemulsions containing supercooled melts are used, one of the systems will have larger droplets and will be at the same or higher temperature than the other supercooled microemulsion. If crystallisation is observed to occur in the microemulsion systems with larger droplet sizes in the course of, typically, several days to weeks, for example between about 2 days and about 4 weeks, but not in the microemulsions with smaller droplet sizes, then the latter systems are shown to contain supersaturated solutions/supercooled melts that are stabilised solely due to confinement. Crystallisation under thermodynamic control can then be effected in these microemulsion systems by either adding more crystallisable material to the microemulsion droplets or by increasing the supersaturation/supercooling slowly and/or slightly, e.g. slowly, as described below.

In certain embodiments of the invention, it may, be appropriate to prepare two identical supersaturated solutions, or supercooled melts, e.g. by dividing an initial system into two parts. These are referred to herein as system A and system B. These putative supersaturated solutions or supercooled melts may be comprised within any of the systems described herein, for example microemulsions, nanoemulsions or liposomes. The test described above, if conducted, to verify absence of prior crystallisation is described as being carried out on system A although it will be understood that this labelling is arbitrary.

Once, or if, the absence of pre-existing crystalline material in system A has been demonstrated, the system B comprising a stabilised supersaturated solution or supercooled melt may then be tested in order to establish whether or not the stability of the confined supersaturated solution or supercooled melt is stabilised solely by virtue of confinement. This may be achieved in a number of ways, in which, typically, the presence of additional crystallisable material at a lower concentration is shown to result (counterintuitively) in crystallisation.

Firstly, in respect of solute crystallisations effected within microemulsions or nanoemulsions, additional crystallising material at a lower concentration in the same solvent (or solvent mixture) to the supersaturated system may be introduced and also at the same temperature. Addition of this additional lower concentration material can be achieved by drop-wise addition (or any other convenient method) with gentle agitation in order to allow the new material entering the dispersed phase access to the discontinuous phase. Conveniently, an identical system to which additional crystallising material has not been added may be agitated using the same force (e.g. by stirring, application of ultrasound, or other methods known to the skilled person) in order to verify that the agitation per se is not influencing or determining the outcome from the system to which the additional material has been added. Also, a layer of the additional amount (e.g. solution) of crystallisable material should not be generated in the system under test. If such a layer is added or formed, this opens up the possibility for crystallisation at the interfacial boundary of the layer with the system (e.g. micro- or nanoemulsion), i.e. at the interface between the newly added material and the existing system, rather than in the confined regions within the system. Any such interfacial crystallisation invalidates the test (i.e. for the supersaturated system B being stabilised solely due to confinement) since any such interfacial crystallisation will not be, by definition, under confinement and, consequentially, not under thermodynamic control.

It will be appreciated, at this point, that it may, in certain embodiments of the invention, be convenient for an initial system, in the confined cavities of which it is desired to crystallise a compound (or co-crystallise more than one compound), to be divided into three aliquots: one (herein System A) to demonstrate the absence of prior crystallisation and so that the system is actually a stabilised supersaturated solution; one (herein System B) to demonstrate that the system is stabilised solely by being confined; and one (herein System C) on which to practice a method of the invention, i.e. actually effecting a crystallisation under thermodynamic control.

Following the addition of the extra crystallisable material to System B, both systems (i.e. System B to which the extra material has been added and System C to which no extra material has been added) may be left to stand, typically for a period of several days or more (for example between about 2 days and about 4 weeks) to verify that system B crystallises, whereas System C does not. Thereafter System C may be crystallised under thermodynamic control by either adding extra crystallisable material, or by increasing the supersaturation by either cooling or the slow addition of anti-solvent. A test applicable for crystallisation from melts within micro-emulsions, nanoemulsions or other confined systems involves addition of additional molten material at a higher temperature. As with the first test above, addition is achieved by way of drop-wise addition with concomitant gentle agitation, gentle so as to avoid interfacial crystallisation and a false positive respectfully.

Material within either a supersaturated solution or supercooled melt stabilised solely by being confined is crystallised under confinement and under thermodynamic control according to the method of the present invention.

In certain embodiments of the invention, crystallisation under confinement and under thermodynamic control is achieved by increasing the degree of supersaturation within the confined system, or amount of material within the confined supercooled melt. Increase in the degree of supersaturation within a confined system may be achieved by one or more methods, for example: (i) a defined cooling regime; (ii) addition of anti-solvent or poorer solvent, e.g. by vapour diffusion, addition of a microemulsion or nanoemulsion or emulsion containing the antisolvent in its confined volumes, dropwise addition or via addition to an emulsified micro- or nanoemulsion; (iii) a chemical reaction; (iv) evaporation of solvent; or (v) or addition of more crystallisable material, typically (i), (ii) or (v).

Typically, the degree of supersaturation within the confined system (i.e. solution), or amount of material (i.e. compound) within the confined supercooled melt, is increased incrementally. In this way, crystallisation takes place within the confined system (as opposed, for example, to interfacial crystallisation) and under thermodynamic control. By incremental is meant a gradual increase in the degree of supersaturation within the confined system, or amount of material within the confined supercooled melt, i.e. not moving immediately or suddenly from one temperature to another, e.g. much lower, temperature. Typically, incrementally changing the temperature refers to a temperature change of about 10° C. to about 0.01° C. per hour or less, for example a temperature change of about 5° C. to about 0.1° C. per hour, such as a temperature change of about 2° C. to about 0.1° C. per hour. Incrementally increasing the amount of material in the droplet means increasing the amount by not more than about 10% (e.g. from about 0.01 to about 5%, or from about 0.01 to 1%) at a time (i.e. per addition of material). Incrementally increasing the supersaturation in the droplet means by not more than about 10% (e.g. from about 0.01 to about 10%, or from about 0.01 to about 5%) of its supersaturation ratio value per hour, or alternatively, at a time.

Alternatively, step-changes to the degree of supersaturation within the confined system, or amount of material within the confined supercooled melt may be effected when crystallising, provided of course that the crystallising occurs under confinement and under thermodynamic control. Thermodynamic control can be assumed to have occurred provided the crystalline product comprises the most stable polymorph, with this outcome not being attributable to the surfactant or co-surfactants used, and use of smaller changes to the supersaturation that fall within the incremental change regime outlined above results in production of this same polymorph, or no crystalline product at all. The method of crystallisation of the invention may be continued until crystals of the desired size are obtained. Alternatively, the crystals formed may be used to seed additional crystallisations (i.e. not according to the invention). This may be achieved by seeding in a supercooled melt or supersaturated solution comprising the same material or by slurrying the crystal obtained by the method of the invention with other solid forms of the same material. These other solid forms may be either amorphous or crystalline; any crystalline forms will convert to the polymorph obtained according to the method of the invention in the case of homogeneous nucleation (i.e. where the surfactant or co-surfactants used to make the microemulsion do not help nucleate the polymorph), since this will be the thermodynamically most stable polymorph. The only exception to this is the rare, but not impossible case, where a phase inversion of stability occurs between the size of the smallest stable nuclei and the bulk phase. The likelihood of such a size-induced stability inversion will become negligible if the smallest stable nuclei contains several hundred molecules or more.

It is important to recognise that the references made herein to systems A to C and the descriptions of various tests prior to the crystallisation method of the present invention are set forth herein in order to fully understand the invention and the provision and manipulation of such systems as described herein are not a mandatory feature of the present invention. Indeed, it is well within the scope of those skilled in the art to develop protocols for practising methods of this invention that do not require intermediate testing to determine, for example, the existence of a supersaturated solution or supersaturated melt stabilised solely by confinement. For instance, having previously determined a supersaturated solution/supercooled melt to be stabilised solely due to confinement in an initial system, a fresh system can be made with incrementally higher supersaturation, or incrementally more crystallisable material compared to the initial system, which can then crystallise under thermodynamic control. For instance, crystallisation of the most stable polymorph under conditions that would produce metastable forms in the analogous unconfined system doped with the same surfactants/co-surfactants provides strong evidence that the crystallisation has been affected under thermodynamic control. However, it is important to repeat that crystallisation in microemulsions, per se, does not lead to thermodynamic control of crystallisation, and so it may still be advantageous to show that a supersaturated solution or supercooled melt that was stabilised solely by confinement occurred prior to crystallisation.

As a corollary of the foregoing, it is to be recognised that, since the present invention is based on the recognition that thermodynamic control over crystallisation is achieved by operating under conditions whereby the nucleation barrier is surmountable, but the amount of material within each confined region is limited, when only a single stable nucleus can form within each region of confinement, with this stable nucleus necessarily comprising the lowest possible energy form(s), it is not necessary for the method and use according to the first and second aspects of this invention to crystallise from a system that is initially a supersaturated solution of supercooled melt comprising the compound the crystallisation of which is desired. Instead a melt that is not initially a supercooled melt, or solution that is not, initially, a supersaturated solution (e.g. an undersaturated or saturated solution), may be used. According to these embodiments of the invention, such initial melts may be treated by cooling or increasing the pressure of the melt; or initial non-supersaturated solutions may be treated by adding more of the compound to and/or increasing the degree of saturation of the solution.

In so doing, crystallising may be achieved under confinement, e.g. nanoconfinement, and under thermodynamic control, in an entirely analogous manner to those embodiments of the invention where in a supersaturated solution or supercooled melt comprising the compound is initially provided, which is stabilised solely due to confinement.

An example of these embodiments of the invention is therefore illustrated by the following, non-limiting steps (described with reference to those embodiments of the invention making use of stabilised, confined supersaturated solutions):
(1) Create an undersaturated solution within a confined volume. This ensures that no nuclei are present;
(2) Relative to a STABILISED supersaturated solution of the same compound within the same confined volume as (1), add more compound, typically incrementally, to the confined volume and/or exceed the concentration of the compound required for supersaturation to induce crystallisation in the confined volume.
(3) ALWAYS achieve thermodynamically most stable polymorph/crystals with improved quality/stability.

It will be understood that the example described above of an embodiment according to this aspect of the invention is exemplary. Crystallisation under thermodynamic control may be effected in undersaturated systems, e.g. microemulsions, by either adding more crystallisable material to the microemulsion droplets or by increasing the supersaturation/supercooling to greater than that of a corresponding supersaturated solution/supercooled melt stabilised due to confinement, as described above. This is achieved, typically, by an incremental increase of the degree of saturation of the compound in the confined solution, relative to a corresponding supersaturated solution of the same compound, which is stabilised solely by being confined; and/or by an incremental increase of the amount of the compound within the confined melt of the same compound, which is stabilised solely by being confined.

An example of a way of increasing the degree of saturation (i.e., defining “degree of saturation” generally, the extent to which any given solution is fully saturated) of the compound in an undersaturated or saturated solution without adding more of the compound is to add antisolvent. In this way the degree of saturation may be increased, for example, by contacting a first microemulsion containing undersaturated solutions of the compound within its dispersed phase and a second microemulsion containing antisolvent for the compound within its dispersed phase, typically whereby to afford a second confined solution of the compound having incrementally or non-incrementally greater supersaturation than the first confined solution of the compound, whereby to allow crystallising under confinement and under thermodynamic control.

An incremental increase in this context may be a temperature change of between 0.01° C. to about 10° C., for example a temperature change of about 0.1° C. to about 0.5° C., such as a temperature change of about 0.1° C. to about 0.2° C.; or an increase in the amount of material in the volume of confinement of not more than about 10% (e.g. from about 0.01 to about 5%, or from about 0.01 to 1%); or an increase of not more than about 10% (e.g. from about 0.01 to about 10%, or from about 0.01 to about 5%) of the degree of supersaturation in the volume of confinement, these increases being relative to a corresponding supersaturated solution of the same compound, which is stabilised solely by being confined; or to a corresponding supercooled melt of the same compound, which is stabilised solely by being confined.

It is important to note that such incremental increases (i.e. relative to a corresponding supersaturated solution of the same compound, which is stabilised solely by being confined; or to a corresponding supercooled melt of the same compound, which is stabilised solely by being confined) may be achieved either incrementally and/or non-incrementally with respect to initially undersaturated solutions or non-supercooled melts, where used. In other words these may, for example, be converted to supersaturated solutions or supercooled melts stabilised solely by confinement by incremental and/or non-incremental (e.g. by step changes) increase to the initial degree of saturation and/or concentration of an undersaturated solution; or incremental and/or non-incremental (e.g. by step changes) cooling or increase to the pressure of the melt. According to some embodiments, therefore, undersaturated solutions or non-supercooled melts may be converted to supersaturated solutions or supercooled melts stabilised solely by confinement by an initial non-incremental increase to the initial degree of saturation and/or concentration of the solution; or non-incremental cooling or increase to the pressure of the melt, whereby to provide a supersaturated solution or supercooled melt stabilised solely by confinement. The attainment of such a system may be achieved by prior development of a protocol to make a supersaturated system stabilised solely by confinement, from a confined undersaturated solution, e.g. as described herein. The degree of supersaturation within such a confined system, or amount of material within the confined supercooled melt may then be increased (e.g. incrementally) as described herein.

According to some embodiments, therefore, undersaturated or saturated, e.g. undersaturated, solutions or non-supercooled melts may be converted to supersaturated solutions or supercooled melts with incrementally more material or incrementally more supersaturation than supersaturated solutions or supercooled melts stabilised solely by confinement, by an initial non-incremental increase to the initial degree of saturation and/or concentration of the solution; or non-incremental cooling or increase to the pressure of the melt, whereby to provide a supersaturated solution or supercooled melt with incrementally more material or supersaturation than a supersaturated solution or supercooled melt stabilised solely by confinement, which thereby crystallises under thermodynamic control. The attainment of such a system may be achieved by prior development of a protocol to make a supersaturated system stabilised solely by confinement, from a confined undersaturated solution, e.g. as described herein.

By ensuring crystallisation in the confined volumes is under thermodynamic control, the crystallisation will produce the thermodynamically most stable product(s) (including the thermodynamically most stable polymorph) in ratios determined by their relative Boltzmann factors for the size particle and confinement conditions (i.e. whether the crystallite is surrounded by solution, or ends up being stabilised by adsorption of surfactant). This can be used to (i) identify the most stable polymorph(s) for homogeneous nucleation (shown by the crystallisation always proceeding at a lower temperature than the homogeneous nucleation temperature for that system), (ii) for heterogeneous nucleation (shown by the crystallisation always proceeding at a higher temperature than the homogeneous nucleation temperature for that system and/or by the surfactants/co-surfactants causing preferential crystallisation of the same material at the planar air-water or oil-water interface), the stabilisation of metastable polymorphs, (iii) for organic and inorganic materials, including proteins and photonic and semiconductor materials, the production of improved perfection crystals, e.g. nanocrystals. Crystals, e.g. nanocrystals may be of organic or inorganic material, and may encompass biologically active molecules such as proteins. Such nanocrystals have utility in a wide variety of applications, for example quantum dots and in the preparation of pharmaceuticals.

The production of high quality protein crystals suitable for structural analysis by X-ray diffraction is particularly important, since understanding the function of a protein requires its structure to be known. Advantageously, however, the present invention provides a generic method for producing the required high quality protein crystals.

In the case of homogeneous nucleation, the most stable polymorph for that size particle is obtainable from the method of the invention. Less stable polymorphs may also be present (i.e. in measurable amounts) if their energies are sufficiently close to that of the most stable polymorph. For instance, consideration of the Boltzmann factor shows that two polymorphs whose confined stable nuclei differ in free energy by a small amount such as 0.2 kJ/mol will result in 52% of the crystalline product being of the more stable form at 300 K, whereas if the most stable form is more stable than all other forms by 10 kJ/mol, the crystalline product will consist essentially of this most stable form. In this way it is possible to identify all low energy polymorphs that may be suitable for drug formulations, for example. It is important to note that the relative stabilities of the nuclei may differ from that of the bulk polymorphs, particularly if these nuclei contain only a few molecules. For instance, for glycine where the γ-polymorph is only ˜0.2 kJ/mol more stable than the α-polymorph in the bulk phases, the proportion of the γ-polymorph that arises can be increased according to the present invention by using microemulsion droplets that contain only a few (typically 3-20) glycine molecules. TEM (transmission electron microscopy) provides a useful method of distinguishing the individual polymorphs that are present, e.g. in nanocrystals, whilst if more than one polymorph is obtained, identification of the most stable form can be established by the usual slurry method. In the case of heterogeneous nucleation, i.e. where the surfactant or co-surfactant is able to aid the nucleation, then polymorphs that are metastable in the bulk may crystallise solely, or in combination with the more stable form(s), depending upon the extent to which the surfactant/co-surfactant can stabilise the less stable form.

A particular advantage of the present invention arises from the ability to provide high-quality crystals and also crystals that may be otherwise difficult to produce, such as proteins. For instance, supersaturated solutions of some materials that are hard to crystallise phase separate into regions of more and less concentrated solutions before crystallisation can occur. This phase separation is followed by the rapid formation of amorphous or poorly crystalline product from the newly-formed, more concentrated solution regions. This is known as oiling out. Performing crystallisation of such materials under thermodynamic control according to the present invention by using confinement will hinder both the phase separation and production of amorphous or poorly crystalline product, since it will not be thermodynamically favourable for either of these events to occur. Thus the present invention allows for crystallisation of such compounds.

The invention may be further understood with regard to the following non-limiting clauses:

1. A method of crystallising a compound comprising providing a supersaturated solution or supercooled melt comprising the compound, the supersaturated solution or supercooled melt being stabilised solely by being confined and then adding more of the material and/or increasing the degree of supersaturation of the supersaturated solution, or cooling or increasing the pressure of the supercooled melt, whereby to effect the crystallising under confinement and under thermodynamic control.

2. The method of clause 1 further comprising demonstrating that the supersaturated solution or supercooled melt is stabilised solely by being confined prior to said crystallising.

3. The method of clause 1 or clause 2 wherein the confined supersaturated solution or supercooled melt is present in a microemulsion, a nanoemulsion or a liposome.

4. The method of clause 3 wherein the confined supersaturated solution or supercooled melt is present in a microemulsion or a nanoemulsion, for example in a microemulsion.

5. The method of any one preceding clause wherein the confined supersaturated solution or supercooled melt is stable for at least one week.

6. The method of any one preceding clause wherein the crystallising is achieved by incremental increase of the degree of supersaturation within a confined supersaturated solution, or amount of material within a confined supercooled melt.

7. The method of clause 6 wherein the crystallising is achieved by incrementally changing the temperature by about 10° C. to about 0.01° C. per hour or less or incrementally increasing the amount of the compound within the supersaturated solution or supercooled melt by not more than about 10% (e.g. from about 0.01 to about 5%) at a time, or incrementally increasing the supersaturation in a supersaturated solution by not more than about 10% of its supersaturation ratio value per hour.

8. The method of any one preceding clause further comprising using the resultant crystals as seeds in a supercooled melt or supersaturated solution comprising the same compound from which the crystals are formed or by slurrying the resultant crystals with other solid forms of the same compound.

9. Use of a supersaturated solution or supercooled melt comprising a compound, the supersaturated solution or supercooled melt being stabilised solely by being confined, in a method of crystallising the material comprising adding more of the compound and/or increasing the degree of supersaturation of the supersaturated solution, or cooling or increasing the pressure of the supercooled melt, whereby to effect the crystallising under confinement and under thermodynamic control.

The invention is illustrated by the following non-limiting examples:

### ROY, Glycine, Mefenamic Acid and Albumin

5-Methyl-2-[(2-nitrophenyl) amino]-3-thiophenecarbonitrile is known as ROY because of its red, orange and yellow polymorphs. ROY is highly polymorphic; to-date ten polymorphs are known (L. Yu, CrystEngComm, 2007, 9, 847) with polymorphic control difficult to obtain from bulk solution without additives to nucleate specific forms (C. A. Mitchell, L. Yu, M. D. Ward, J. Am. Chem. Soc. 2001, 123, 10830; C. P. Price, A. L. Grzesiak, A. J. Matzger, J. Am. Chem. Soc. 2005, 172, 5512). At ambient temperatures, the most stable yellow prism form is ˜300 J mol⁻¹ lower in energy than the next known stable phases, the orange needle and orange prism forms (S. Chen, H. Xi, L. Yu, J. Am. Chem. Soc. 2005, 127, 17439; L. Yu, G. A. Stephenson, C. A. Mitchell, C. A. Bunnell, S. V. Snorek, J. J. Bowyer, T. B. Borchardt, J. G. Stowell, S. R. Byrn J. Am. Chem. Soc. 2000, 122, 585). However these forms are enantiomorphic, and the orange needle form becomes the most stable phase above 70° C. By using small volumes of ˜1 to 50 μl in the capillary precipitation of ROY at ambient temperatures, the higher energy orange needle polymorph of ROY can be favoured over the stable yellow prism form due to the high supersaturation values achieved (J. L. Hilden, C. E. Reyes, M. J. Kelm, J. S. Tan, J. G. Stowell, K. R. Morris Cryst. Growth Des. 2003, 3, 921).

The present invention uses much smaller confinements, e.g. approximately 0.01 to 10's fl (femtolitres), so that the crystallisation is under thermodynamic rather than kinetic control due to the scarcity of material within each droplet. In this way high degrees of supersaturation are achieved but still the most stable polymorph is obtained. The metastable red prism form of ROY could be obtained selectively by crystallisation within nanoporous polycyclohexylethylene (PCHE) monoliths (J- M Ha, J. H. Wolf, M. A. Hillmyer, M. D. Ward, J. Am. Chem. Soc. 2004, 126, 3382). The red prism nanocrystals were aligned with the pore diameter suggesting heterogeneous nucleation and so in this case crystallisation of a metastable form was favoured by the large surface area of PCHE in the nanoporous material. Notably, the techniques described by Ha et al. differ from those of the present invention in two respects, firstly the aim is to use the large surface area to volume of nanopores to obtain metastable forms by a specific interaction between the metastable form and the nanopore walls, and secondly, the nanopores are not closed, and hence the crystallisation occurs in restricted but not confined volumes, as defined herein.

Glycine has four polymorphs. Three of these, the α-, β-, and γ-forms exist at ambient pressure, whilst the fourth, the δ-form, can be obtained under applied pressure. Of the ambient pressure forms, the γ-phase is the most stable at temperatures below ˜165° C., with the a-phase the most stable at higher temperatures. The β-phase is the least stable. At ambient temperatures the γ-phase is ˜200 J mol⁻¹ and ˜600 J mol⁻¹ lower in energy than the α- and β-phases, respectively (G. L. Perlovich, L. K. Hansen, A. Bauer-Brandl, J. Therm. Anal. Cal. 2001, 66, 699; E. V. Boldyreva, T. N. Drebushchak, T. P. Shakhtshneider, H. Sowa, H. Ahsbahs, S. V. Goryainov, S. N. Ivashevskaya, E. N. Kolesnik, V. A. Drebushchak, E. B. Burgina, ARKIVOC 2004 (xii) 128-155). Despite its slightly greater stability, the γ-phase is difficult to obtain from aqueous solution at neutral pH, and the α-phase typically crystallises. Initially it was thought that a-phase nucleation was favoured due to the dimer growth units of the α-phase pre-existing in aqueous solution. However recent findings have cast doubt on this (J. Huang, T. C. Stringfellow, L. Yu, J. Am. Chem. Soc. 2008, 130, 13973; S. Hamad, C. E. Hughes, C. R. Catlow, K. D. M. Harris J. Phys. Chem. B 2008, 112, 7280.). The a-phase grows ˜500 times more quickly than the γ-phase (J W Chew, S N. Black, P S Chow, R B. H. Tanc and K J. Carpentera, CrystEngComm 2007, 9, 128) and so this also helps explain its prevalence. Crystallisation of γ-glycine in AOT-stabilized microemulsions has been achieved by Gat et al (infra). However, the surfactant AOT is known to induce γ-glycine nucleation at the planar oil-water interface (K. Allen, R. J. Davey, E. Ferrari, C. Towler, G. J. Tiddy Cryst. Growth Des. 2002, 2, 523), and hence the achievement of the most stable form of glycine in these microemulsions is aided by the AOT. The present invention allows crystallisation of the most stable polymorph to be achieved in microemulsion systems where the surfactant does not induce nucleation (i.e. homogeneous nucleation occurs) or in systems where the surfactant aids nucleation of a less stable form.

Mefenamic acid has two polymorphs, form I and form II, with form I being the stable polymorph at ambient temperatures. Crystallization of mefenamic acid from bulk DMF only produces the metastable form II, irrespective of the rate of crystallization or the crystallization temperature employed (A. J. Alvarez et al, (Cryst. Growth Des. 2002, 2, 4181); A. J. Aguiar and J. E. Zelmer, (J. Pharm. Sci. 1969, 58, 983); and S. Cesur and S. Gokbel (Cryst. Res. Technol. 2008, 43, 720)).

Glycine, ROY and mefenamic acid are ideal candidates to demonstrate the present invention and demonstrate how thermodynamic control of crystallisation in confined volumes may be achieved given the difficulty in obtaining, without specific additives, the most stable γ-form of glycine, the difficulty in achieving polymorphic control for ROY, and for mefenamic acid the inability to obtain the most stable Form I from DMF solutions. Albumin is used to exemplify how protein crystallisation can be achieved.

### Preparation of Solutions and Microemulsions

Microemulsions are thermodynamically stable and so the order of mixing, and method of mixing (e.g. vortexing or sonicating) can be changed without altering the final microemulsion state.

### Glycine Solutions

The required amount of glycine was dissolved in water at the required temperature.

### Glycine Microemulsions at Ambient Temperatures

The required amounts of oil (typically heptane), surfactant mix (typically a 1:1 weight ratio of Brij 30 and Span 80), and glycine solution were added to a glass vial, shaken by hand for 10-20 seconds and then vortexed at 2000 rpm for 10 seconds.

### Glycine Microemulsions at 50° C.

The required amounts of oil (typically hexadecane), surfactant mix (typically a 1:1 weight ratio of Span 20 and Span 80), ethanol, and glycine solution, all previously stored at 50° C. or above for at least 3 hours were added to a heated glass vial and sonicated in an ultrasonic water bath at 50° C. for 1 minute. The microemulsion was kept at 50° C. until further use.

### ROY Solutions

The required amount of ROY was dissolved in toluene at ambient temperature.

### ROY Microemulsions at Ambient Temperatures

The required amounts of water, surfactant (typically Igepal 720A), alkane (typically heptane or hexadecane) and ROY solution in toluene were added to a glass vial, shaken by hand for 10-20 seconds and sonicated in an ultrasonic water bath at ambient temperature for 10 seconds.

### Mefenamic Acid Solutions

The required amount of mefenamic acid was dissolved in DMF at 50-60° C.

### Mefenamic Acid Microemulsions

The required amounts of heptane, AOT, and mefenamic acid solution, all previously stored at 50° C. or above for at least 3 hours were added to a heated glass vial and sonicated in an ultrasonic water bath at 50° C. for 1 minute. The microemulsion was kept at 50° C. until further use.

### Egg White Albumin Solutions

The required amounts of egg white albumin was dissolved in water at ambient temperature.

### Egg White Albumin Microemulsions

The required amounts of isooctane, AOT and albumin solution were added to a glass vial, shaken by hand for 10-20 seconds and then vortexed at 2000 rpm for 10 seconds.

### Crystallisation Conditions

Crystallisation in the microemulsion was typically induced by cooling or addition of antisolvent, either by adding a microemulsion or emulsion containing the antisolvent in its dispersed phase, or by drop-wise or via vapour diffusion within sealed containers.

### Analysis of Crystals

Nanocrystals within the microemulsions were analysed by placing drops of the microemulsion onto TEM holey carbon grids and placing in a TEM. Nanocrystal images were taken and selected area electron diffraction patterns from the nanocrystals were captured, from which their polymorphic form could be determined.

Larger crystals or aggregates of crystals were obtained from the microemulsions over extended periods of several days, weeks or months depending upon the system. Crystals/crystal aggregates were typically extracted via filtration. X-ray diffraction and/or FTIR and/or Raman microscopy techniques were used to determine the polymorph(s) of the extracted crystals/crystal aggregates.

### Crystallisation Procedures

### Example 1

Predominantly γ-Glycine crystallisation in microemulsions by adding a second microemulsion containing methanol antisolvent, using surfactants that nucleate the β-polymorph predominantly, with some α-polymorph, in emulsions and at the planar air-water/oil-water interface

A glycine microemulsion was prepared at ambient temperature containing 4.2 g heptane, 2.8 g of a 1:1 mass ratio of Span 80 and Brij 30, and 0.25 g of glycine solution containing 4% by mass of glycine in water. The microemulsion was shaken by hand to ensure homogeneous mixing. To this was added a microemulsion containing 1.8 g heptane, 2.8 g of a 1:1 mass ratio of Span 80 and Brij 30, 1 g of methanol and 0.25 g of water, with subsequent shaking to ensure homogeneous mixing. The resulting supersaturation ratio (c/cₛₐₜ) was 2.3. The resultant microemulsion was left to stand for 3 weeks at 25° C. FTIR analysis on the extracted crystals confirmed that they were predominantly of γ-glycine.

### Example 2

Predominantly y-Glycine crystallisation in microemulsions by cooling, using surfactants that nucleate the β-polymorph predominantly, with some α-polymorph, in emulsions and at the planar air-water/oil-water interface

A glycine microemulsion was prepared at 50° C. containing hexadecane, 15% by mass of a 1:1 mass ratio of Span 20 and Span 80, 1% by volume of ethanol and 1% by volume of a glycine solution containing 7% by mass of glycine,. The microemulsion was placed in a water bath at 50° C. and cooled to 25° C. over 24 hours. TEM analysis after 7 days confirmed the nanocrystals/nanocrystal aggregates were predominantly of γ-glycine.

### Example 3

Predominantly γ-Glycine crystallisation in microemulsions by vapour diffusion of antisolvent, using surfactants that nucleate the α- and β-polymorphs in emulsions and at the planar air-water/oil-water interface 16 g of a glycine microemulsion was prepared at ambient temperatures containing heptane, and 25% by mass of a 1:1 mass ratio of Span 80 and Brij 30, to which was added 0.45 g of a glycine aqueous solution containing 4% by mass glycine. The microemulsion was placed in a glass vial which was placed in a larger glass vessel to which methanol had been added. A cover was placed over the larger glass vessel and the system left at ambient temperature. After a day crystals/crystal aggregates had appeared. FTIR analysis confirmed the crystals/crystal aggregates were predominantly of γ-glycine.

### Example 4

Predominantly stable Form I mefenamic acid crystallization from DMF microemulsions, despite Form II only being produced from bulk DMF solutions.

The mefenamic acid-in-DMF bulk solutions and microemulsions, were prepared at elevated temperature (˜50-60° C.). The microemulsion consisted of 3 g of AOT solution containing 3% by mass of AOT in heptane to which was added 20 μl of a mefenamic acid solution containing 80-100 mg of mefenamic acid per ml DMF. The microemulsion was cooled from 50 to 8° C. over 12 hours and then left 8° C. The resulting supersaturation ratio (c/cₛₐₜ) was 4.4-5.5. The crystals produced using this method were predominantly of Form I, but the time taken to grow ˜0.5 mm³ crystals suitable for Raman microscopy could be prohibitively long (i.e. ˜months). Crystals of ˜0.5 mm³ size could be grown more quickly by adding more microemulsion solution, so that crystals suitable for Raman microscopy could be grown in a week to several weeks. Any unwanted Form II crystals could be removed from the microemulsion by leaving the microemulsion at ambient temperatures.

### Example 5

Yellow prism ROY crystallisation in microemulsions by dropwise addition of antisolvent

ROY microemulsions were prepared at ambient temperatures containing 10% by mass of Igepal CA720 in water and 30 μl/g or 60 μl/g of a ROY in toluene solution containing between 8% to 10% by mass of ROY. Immediate drop-wise addition of 12 μl/g of heptane to the freshly prepared microemulsion followed by shaking by hand for 10-20 seconds and sonication in an ultrasonic water bath at ambient temperature for 10 seconds resulted in a microemulsion state again from which crystallisation occurred. The crystals/crystal aggregates appeared over a few hours to several hours. The crystals/crystal aggregates were extracted by filtration. FTIR analysis confirmed the crystals/crystal aggregates were of yellow prism ROY, the most stable ROY polymorph at ambient temperatures.

### Example 6

An egg white albumin microemulsion was prepared at ambient temperature containing 0.9 g isooctane, 0.1 g AOT, and 0.02 g of the albumin solution containing 5 mg albumin per ml water. The microemulsion was shaken by hand to ensure homogeneous mixing. To this was added a microemulsion containing 0.9 g isooctane, 0.1 g AOT and 0.1 g methanol, with subsequent shaking to ensure homogeneous mixing. The resulting microemulsion was left standing with crystals observed by optical microscopy after 48 hours.

### Examples of Supersaturated Solutions Stabilised Due to Confinement

### Glycine

Glycine microemulsions prepared at ambient temperature containing 4.2 g heptane, 2.8 g of a 1:1 mass ratio of Span 80 and Brij 30, and 0.25 g of glycine solution containing 3-3.5% by mass of glycine in water. The microemulsion was shaken by hand to ensure homogeneous mixing. To this was added a microemulsion containing 1.8 g heptane, 2.8 g of a 1:1 mass ratio of Span 80 and Brij 30, 1 g of methanol and 0.25 g of water, with subsequent shaking to ensure homogeneous mixing. The resulting supersaturation ratio (c/cₛₐₜ) was at 1.7-2.0, i.e. sufficient to cause fast crystallisation of metastable forms in an unconfined system. No crystals were observed by visual inspection or optical microscopy after 3 months of standing for the microemulsion made from 3% by mass of glycine in water, with only a few μm sized crystals apparent in the microemulsion made from 3.5% glycine in water after 3 months.

Glycine microemulsions prepared at 50° C. containing hexadecane, 15% by mass of a 1:1 mass ratio of Span 20 and Span 80, 1% by mass of ethanol and 1% by mass of a glycine aqueous solution containing 2.5% by mass of glycine. The microemulsion was placed in a water bath at 50° C. and cooled to 26° C. over 24 hours. The resulting supersaturation ratio (c/cₛₐₜ) was at least 4, i.e. sufficient to cause rapid crystallisation in an unconfined system. No crystals were observed by visual inspection or optical microscopy after 1 month of standing.

### ROY

ROY microemulsions prepared at ambient temperatures containing 10% by mass of Igepal CA720 in water, 30 μl/g of a toluene solution containing 6% by mass of ROY and 12 μl/g of heptane. The resulting supersaturation ratio (c/cₛₐₜ) was at least 4, i.e. sufficient to cause crystallisation typically within a few hours in an unconfined system. No crystals were observed by visual inspection or optical microscopy after 1 month of standing.

### Mefenamic Acid

The mefenamic acid-in-DMF bulk solutions and microemulsions, were prepared at elevated temperature (˜50-60° C.). The microemulsion consisted of 3 g of AOT solution containing 3% by mass of AOT in heptane to which was added 20 μl of a mefenamic acid solution containing 70-75 mg of mefenamic acid per ml DMF. The microemulsion was cooled from 50 to 8° C. over 12 hours and then left at 8° C. The resulting supersaturation ratio (c/cₛₐₜ) was 3.9-4.1, i.e. sufficient to cause crystallisation of metastable form II typically within a few hours in an unconfined system. No crystals were observed by visual inspection or optical microscopy after 1 month of standing.

1. A method of crystallizing a compound comprising either:
(i) providing a first confined solution comprising the compound; and adding more of the compound to and/or increasing the degree of saturation of the first confined solution, to thereby provide a resultant second confined solution that comprises more compound and/or that has a greater degree of supersaturation relative to a confined supersaturated solution of the same compound stabilized solely by being confined; or
(ii) providing a first confined melt comprising the compound; and cooling and/or increasing the pressure of the first confined melt, to thereby provide a resultant second confined melt that is cooler and/or is more pressurized relative to a confined supercooled melt of the same compound stabilized solely by being confined,
to thereby effect the crystallizing under confinement and under thermodynamic control.

2. The method of claim 1 wherein the crystallizing is effected by incrementally increasing the degree of saturation of the first confined solution, or incrementally cooling or increasing the pressure of the first confined melt.

3. The method of claim 1 wherein the crystallizing is effected by incrementally and/or non-incrementally increasing the degree of saturation of the first confined solution, or incrementally and/or non-incrementally cooling or increasing the pressure of the first confined melt.

4. The method of claim 1, which is a method of crystallizing a compound comprising providing a supersaturated solution or supercooled melt comprising the compound, the supersaturated solution or supercooled melt being stabilized solely by being confined and then adding more of the compound and/or increasing the degree of supersaturation of the supersaturated solution, or cooling or increasing the pressure of the supercooled melt, to thereby effect the crystallizing under confinement and under thermodynamic control.

5. The method of claim 1 comprising either:
(i) providing the first confined solution comprising the compound; and adding more of the compound and/or increasing the degree of saturation of the first confined solution, to thereby provide a supersaturated solution of the compound that is stabilized solely by being confined and then adding more of the compound and/or increasing the degree of supersaturation of the confined supersaturated solution; or
(ii) providing the first confined melt comprising the compound; and cooling and/or increasing the pressure of the first confined melt, to thereby provide a supercooled melt of the compound that is stabilized solely by being confined and/or then cooling or increasing the pressure of the confined supercooled melt,
to thereby effect the crystallizing under confinement and under thermodynamic control.

6. The method of claim 4 further comprising demonstrating that the supersaturated solution or supercooled melt is stabilized solely by being confined prior to said crystallizing.

7. The method of claim 4 wherein the confined supersaturated solution or supercooled melt is present in a microemulsion, a nanoemulsion or a liposome.

8. The method of claim 7 wherein the confined supersaturated solution or supercooled melt is present in a microemulsion or a nanoemulsion.

9. The method of claim 4 wherein the confined supersaturated solution or supercooled melt is stable for at least one week.

10. The method of claim 4 wherein the crystallizing is achieved by incremental increase of the degree of supersaturation within a confined supersaturated solution, or amount of compound within a confined supercooled melt.

11. The method of claim 10 wherein the crystallizing is achieved by incrementally changing the temperature by about 10° C. to about 0.01° C. per hour or less or incrementally increasing the amount of the compound within the supersaturated solution or supercooled melt by not more than about 10% at a time, or incrementally increasing the supersaturation in a supersaturated solution by not more than about 10% of its supersaturation ratio value per hour.

12. The method of claim 1 wherein a first confined solution comprising the compound is provided and the degree of saturation of the first solution with the compound is increased, whereby the second solution has incrementally greater supersaturation than a supersaturated solution of the same compound stabilized solely by being confined.

13. The method of claim 12 wherein the first, second and supersaturated confined solutions are each present in the dispersed phase of a microemulsion, each present in the dispersed phase of a nanoemulsion or each present in a liposome.

14. The method of claim 12 wherein the first, second and supersaturated confined solutions are each present in the dispersed phase of a microemulsion.

15. The method of claim 1, wherein the second confined solution is prepared by introducing antisolvent for the compound into the first confined solution.

16. The method of claim 15 wherein the antisolvent is provided within the dispersed phase of a microemulsion.

17. The method of claim 1 further comprising using the resultant crystals as seeds in a supercooled melt or supersaturated solution comprising the same compound from which the crystals are formed or by slurrying the resultant crystals with other solid forms of the same compound.

18-19. (canceled)
